(19) United States
(12) Patent Application Publication
(10) Pub. No.: US 2006/0129016 A1
(43) Pub. Date: Jun. 15, 2006

(54) Methods for controlling water content of sulfuric acid in a sulfuric acid catalyzed process

(76) Inventors: Mark C. Anderson, Spring, TX (US); Michael F. Ray, Spring Branch, TX (US)

Correspondence Address: GREENLEE WINNER AND SULLIVAN P C, 4875 PEARL EAST CIRCLE, SUITE 200, BOULDER, CO, 80301, US

(21) Appl. No.: 11/009,996

(22) Filed: Dec. 10, 2004

(51) Int. Cl. C07C 2/58

(52) U.S. Cl. 585/731

(57) ABSTRACT

A method is provided for reducing the water content of a sulfuric acid catalyst in a sulfuric acid-catalyzed process carried out in a reactor comprising: (a) withdrawing a portion of catalyst from the acid settler, forming a withdrawn catalyst stream; (b) continuously adding an SO₃-containing substance to the withdrawn catalyst stream at a desired rate, forming a fortified catalyst, while maintaining the temperature of the fortified catalyst stream below about 60° F.; (c) returning the fortified catalyst to the reactor; whereby the water concentration in the fortified catalyst is maintained at 1.5 to 4 weight percent of the catalyst. A method is also provided for drying paraffinic feed or recycle hydrocarbon streams in a reactor system comprising contacting the feed or recycle hydrocarbon streams with spent sulfuric acid, whereby the feed or recycle streams are dried and whereby a portion of the sulfuric acid esters in the spent acid are converted to sulfuric acid and alkylate. The alkylate produced is extracted into the hydrocarbon phase and returns to the reactor system.

### BACKGROUND OF THE INVENTION

Various processes of industrial significance are catalyzed by concentrated sulfuric acid. In some such processes, the sulfuric acid becomes diluted with water that enters the process incidentally, intentionally or inadvertently, or is a product of the reaction. This dilution causes the sulfuric acid catalyst to become ineffective, corrosive, or otherwise unusable. Currently, to keep the acid concentration high enough, a portion of the sulfuric acid catalyst is withdrawn from the process, either continuously or intermittently, and replaced with fresh sulfuric acid having a concentration typically from 98.0 to 99.6% H₂SO₄. Both the disposal of the spent acid and the replacement with fresh acid represent significant costs of operation.

One sulfuric acid-catalyzed process is sulfuric acid alkylation, in which a feed stream comprising one or more isoparaffins, preferably containing 4 to 5 carbon atoms is contacted with an alkylating agent and sulfuric acid catalyst. The alkylating agent comprises one or more olefins, preferably containing 3 to 5 carbon atoms. In the sulfuric acid alkylation process, the rate of acid replacement is usually adjusted to limit catalyst dilution with both acid-soluble hydrocarbon polymer (“red oil”) produced and water. Red oil is a hydrocarbonaceous polymer produced in the alkylation reaction. It is soluble in the acid phase and has been shown to be beneficial as a component of the catalyst up to a concentration of about 4-20% by weight of the acid phase. If present in the acid above an optimum concentration of about 1.5 to 4 weight % of the acid phase, water reduces the catalytic function of the acid, lowers the solubility of hydrocarbons in the acid, and increases the corrosivity of the acid to the process equipment itself. Because of the cost to replace acid, typically two to four cents per gallon of alkylate, sulfuric acid alkylation units are often operated with a higher concentration of water in the acid than is optimum for product quality, specifically the octane number of the alkylate. Furthermore, many alkylation units operate at a concentration of red oil below the optimum because dilution of the acid with water forces the acid to be replaced at a higher rate than required to maintain red oil at a desired higher concentration. In typical practice, the acid is replaced at a rate chosen to maintain the sum of the concentrations of diluents (water plus red oil) in the acid phase at about 10-13%.

Various methods have been proposed to reduce the rate of acid replacement necessary in a sulfuric acid-catalyzed process, either by reducing the dilution of the acid with red oil or by reducing dilution of the acid with water. U.S. Pat. No. 6,007,722 (Dec. 28, 1999) reports a method for extracting organic impurities from a sulfuric acid phase containing at least about 70% by weight sulfuric acid using supercritical or liquid carbon dioxide. U.S. Pat. No. 5,095,168 (Mar. 10, 1992) discloses a process to reduce the rate of polymer formation in the alkylation reaction to react C3-C5 olefins with isobutane in the presence of a sulfuric acid catalyst by maintaining a process temperature of between 20 and 30 degrees F. Methods to reduce dilution of the acid with water include reducing the concentration of water in the feed streams or removing it from the acid phase. The streams entering the reactor may contain both undissolved and dissolved water. In current processes, undissolved water in one or more hydrocarbon streams entering the reactor is reduced by physical means such as coalescing filters, sand bed coalescing, or salt drying. Several means have been proposed to remove dissolved water, including the use of regenerable solid or liquid dessicants known in the art, such as glycol. However, the use of these conventional drying agents incurs a substantial cost to regenerate the drying agent. U.S. Pat. No. 4,677,245 (Jun. 30, 1987) discloses an alkylation process wherein the feed hydrocarbons are dried using a fractionation column or absorbent drying to reduce the rate of water ingress into the unit so that the water concentration of the alkylation catalyst can be maintained to less than 2 weight percent at lower acid replacement rate than otherwise possible. U.S. Pat. No. 4,677,245 does not discuss another major source of water entering the reaction, namely the recycle isobutane stream. U.S. Pat. No. 6,159,382 (Dec. 12, 2000) reports a method for rejecting water from a sulfuric acid solution containing from 10 to 95% by weight sulfuric acid by cooling the sulfuric acid solution near the freezing point of the solution to form a slurry of acid-rich and acid-poor regions that are separated on the basis of density. U.S. Pat. No. 5,547,655 (Aug. 20, 1996) reports an electrochemical and photolytic process at elevated temperature for removing water from spent sulfuric acid catalyst from the alkylation of C3-C5 olefins and alkanes. U.S. Pat. No. 5,888,920 (Mar. 30, 1999) also describes an electrolytic process for regenerating an aqueous sulfuric acid phase from an alkylation process. The electrolytic removal of water, using an electrical current to convert water to hydrogen and oxygen, is prohibitively expensive.

Other patents disclose processes aimed at reacting the water in the acid phase with SO₃ to form additional sulfuric acid: U.S. Pat. No. 4,148,836 (Apr. 10, 1979) reports a method for periodic fortification of sulfuric acid catalyst with sulfur trioxide fortifying agents in a process for alkylating C4-C5 isoparaffin feed stocks with C2-C5 olefins in the presence of a concentrated sulfuric acid catalyst containing at least 1% but less than 4% water, followed by cooling the acid catalyst, followed by a delay in re-introducing the catalyst into the process. The process described in U.S. Pat. No. 4,148,836 specifies that the fortification must be performed discontinuously, during less than 6% of the time the sulfuric acid is in contact with the hydrocarbons in the alkylation zone of the process. U.S. Pat. No. 4,148,836 further describes removing volatile organic impurities in the acid catalyst prior to addition of sulfur trioxide.

U.S. Pat. No. 4,260,846 (Apr. 7, 1981) reports a method for periodic fortification of sulfuric acid catalyst with a sulfur trioxide-bearing fortifying agent in a process for the alkylation of isoparaffins with olefins. However, like U.S. Pat. No. 4,148,836, the process described in U.S. Pat. No. 4,260,846 specifies periodic, discontinuous addition of oleum to fortify the circulating sulfuric acid catalyst. Both U.S. Pat. Nos. 4,148,836 and 4,260,846 specify addition of the oleum during some fraction of the time the acid is in contact with the hydrocarbon, but in a commercial unit, where the acid is continuously recirculated and is in continuous contact with the hydrocarbon, that parameter is meaningless.

The processes that react the free water in the acid phase with SO₃ to produce sulfuric acid are seldom if ever practiced commercially. In an attempt to understand why such allegedly valuable technology is not practiced, the inventors of the present invention conducted bench scale testing that demonstrated that when practiced as taught by the previous patents, side reactions produce undesirable coproducts whose formation consumes SO₃. These coproducts can also slow down separation of the acid and hydrocarbon phases, which separation is essential to the alkylation process.

There remains a need in the art for an improved method of reducing the water content of a sulfuric acid catalyst in a sulfuric acid-catalyzed process without adversely affecting the performance of the alkylation process.

### BRIEF SUMMARY OF THE INVENTION

The present invention describes two methods to reduce the water content of sulfuric acid in a sulfuric acid-catalyzed process. These methods may be used individually or in combination. The methods of the invention may also be used with currently used procedures to reduce water in a sulfuric acid-catalyzed process.

The first method to reduce the water content of sulfuric acid in a sulfuric acid-catalyzed process reduces the introduction of water into the reaction section of the process by removing water from the paraffinic hydrocarbon streams fed to the reactor. In general, it is preferred that all hydrocarbon streams entering the reactor be first dewatered by physical means such as a gravimetric phase separation, for example a coalescer/settler, as known in the art. The present invention uses spent acid to dry one or more paraffinic hydrocarbon streams entering the reactor, which includes the recycle isobutane stream as well as any paraffinic hydrocarbon stream, to reduce dissolved water.

As used herein, “spent acid” means the acid concentration is lower than the acid concentration in pure acid. In one preferred process, “spent acid” means the acid withdrawn from the acid settler for disposal. As used herein, “reduce” means lower the concentration of a specified substance. “Reduce” may mean eliminate, but any measurable lowering is considered to be encompassed in the term “reduce”. As used herein, “dewater” means to reduce undissolved water in a stream, such as a hydrocarbon stream by physical means, such as gravimetric phase separation. “Dehydrate” or “dry” means reduce the concentration of dissolved water in a stream, such as a hydrocarbon.

The second method to reduce the water content of sulfuric acid in a sulfuric acid-catalyzed process is to use continuous acid fortification with an SO₃-containing substance instead of, or in addition to, commercial grade or other grades of sulfuric acid as catalyst makeup. The SO₃ reacts with water in the acid to produce sulfuric acid according to the reaction:
SO₃+H₂O→H₂SO₄
Continuous acid fortification using the description and under the conditions disclosed herein avoids or minimizes the undesired reaction of SO₃ with hydrocarbons that produce undesirable coproducts, as described elsewhere herein.

The methods may be used separately, or in combination.

More particularly, provided is a method for reducing water from one or more hydrocarbon streams consisting essentially of paraffins (paraffinic hydrocarbon streams) before the one or more streams contact the acid catalyst of a process catalyzed by sulfuric acid carried out in a reactor (such as an alkylation reactor), comprising:
(a) removing a stream of spent acid from the reactor at a rate modulated to control the concentration of red oil in the acid catalyst in the reactor system;
(b) contacting the one or more paraffinic hydrocarbon streams with the spent acid in one or more drying contactors, forming dried paraffinic hydrocarbon stream(s) and an acid phase;
(c) separating the dried paraffinic hydrocarbon stream(s) from the acid phase(s) in the drying separator(s);
(d) sending the dried paraffinic hydrocarbon stream(s) to the reactor;
(e) removing the acid phase(s) from the drying separator(s) to regeneration or disposal.

The paraffinic hydrocarbon streams may be treated separately or combined before drying. “Paraffinic hydrocarbon” means any alkane, including mixtures of chain length. In one embodiment, the hydrocarbon stream is a recycle isoparaffin stream.

Also provided is a method for reducing the water content of a sulfuric acid catalyst in a sulfuric acid-catalyzed process carried out in a reactor (such as an alkylation reactor) comprising: (a) continuously circulating a stream of acid catalyst substantially free of hydrocarbons from the reactor system to a mixing device, forming a wet catalyst stream; (b) in the mixing device, continuously adding an SO₃-containing substance to the wet catalyst stream at a rate calculated to convert water in the circulating catalyst stream to sulfuric acid at a desired rate, forming a fortified catalyst stream, while maintaining the temperature of the wet catalyst stream and fortified catalyst stream below about 60° F., wherein the rate of circulation of the catalyst stream is selected to control the concentration of water in the fortified catalyst stream to at least about 0.8% by weight of the acid and the rate of addition of SO₃-containing substance is selected to control the concentration of water at one or more zones in the reactor to a selected value; (c) returning the fortified catalyst stream to the reactor, preferably at a point that provides mixing of the fortified catalyst stream with the bulk of the circulating acid catalyst before mixing with hydrocarbon feed, whereby the water content of the catalyst in the reactor is maintained at a selected value, preferably about 1.5 to 4% by weight of acid.

In order to avoid undesired side reactions between the SO₃ and the red oil present in the circulating catalyst, the rate of circulation of wet catalyst from the reactor to react with the SO₃-containing substance must be high enough that the resulting fortified catalyst stream returned to the reactor contains at least 0.8% water by weight of acid.

In one embodiment, the alkyation process comprises two or more separate reactor systems with different hydrocarbon feed streams, each reactor having a separate acid settler, wherein catalyst streams are withdrawn from separate acid settlers, fortified separately, and returned to points in each reactor system to optimize the concentration of water in the separate alkylation reactors. In one embodiment, the SO₃-containing substance is oleum.

In another example, provided is a method for reducing water in a sulfuric acid catalyst in a sulfuric acid-catalyzed process carried out in a reactor (such as an alkylation reactor) comprising: (a) continuously circulating a stream of acid catalyst substantially free of hydrocarbons from the reactor system to a mixing device, forming a wet catalyst stream; (b) in the mixing device, continuously adding an SO₃-containing substance to the wet catalyst stream at a rate calculated to convert water in the circulating catalyst stream to sulfuric acid at a desired rate, forming a fortified catalyst stream, while maintaining the temperature of the wet catalyst stream and fortified catalyst stream below about 60° F., wherein the rate of circulation of the catalyst stream is selected to control the concentration of water in the fortified catalyst stream to at least about 0.8% by weight of the acid and the rate of addition of SO₃-containing substance is selected to control the concentration of water at one or more zones in the reactor to a selected value; (c) returning the fortified catalyst stream to the reactor, preferably at a point that provides mixing of the fortified catalyst stream with the bulk of the circulating acid catalyst before mixing with hydrocarbon feed, whereby the water content of the catalyst in the reactor is maintained at a selected value, preferably about 1.5 to 4% by weight of acid, (d) removing spent acid from the reactor; contacting one or more hydrocarbon streams with the spent acid in a drying contactor, forming one or more dried hydrocarbon streams and an acid phase; (e) separating the dried hydrocarbon streams from the acid phase in a drying separator; (f) passing the dried hydrocarbon streams to the reactor; (g) removing the acid phase from the drying separator to disposal or regeneration. In one embodiment, the acid catalyst in the reactor after return of the fortified catalyst stream contains between 1.5 to 4% water by weight. In one embodiment, the SO₃-containing substance is oleum.

One of ordinary skill in the art will appreciate that methods, order of steps, device elements, starting materials, synthetic methods, and process steps other than those specifically exemplified can be employed in the practice of the invention without resort to undue experimentation. All art-known functional equivalents, of any such methods, order of steps, device elements, starting materials, synthetic methods, and process steps are intended to be included in this invention. Whenever a range is given in the specification, for example, a temperature range, a time range, or a concentration range, all intermediate ranges and subranges, as well as all individual values included in the ranges given are intended to be specifically and individually included in the disclosure.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. As used herein, “substantially” takes the same meaning as consisting essentially of. Any recitation herein of the term “comprising”, particularly in a description of components of a composition or in a description of elements of a device, is understood to encompass those compositions and methods consisting essentially of and consisting of the recited components or elements. The invention illustratively described herein suitably may be practiced in the absence of any element or elements, limitation or limitations which is not specifically disclosed herein.

In general the terms and phrases used herein have their art-recognized meaning, which can be found by reference to standard texts, journal references and contexts known to those skilled in the art. The definitions are provided to clarify their specific use in the context of the invention.

### BRIEF DESCRIPTION OF THE DRAWINGS

FIG. 1 shows one embodiment of an alkylation process.

FIG. 2 shows one embodiment of acid drying of reactant streams entering an alkylation reactor and acid fortification with an SO₃-containing substance.

### DETAILED DESCRIPTION OF THE INVENTION

The present invention offers many advantages over conventional processes. Although a sulfuric acid-catalyzed alkylation process is specifically described, the methods of the invention may be used in any process that is catalyzed by sulfuric acid. Certain process parameters are not specifically described herein, but are known to one of ordinary skill in the art.

In the present invention, water contaminating the sulfuric acid catalyst is reduced in two ways: first, water in streams entering the reactor (feed streams) is reduced by dehydrating at least a portion of one or more feed streams using spent acid as a drying agent; and second, water in the catalyst itself is reduced by reaction with a SO₃-containing material under conditions that control the temperature of the fortified acid to below about 60° F. and that do not reduce the water in the fortified acid to less than about 0.8%. The processes may be used separately or in combination. The processes of the invention may be used in combination with conventional drying methods.

The invention may be further understood by reference to the Figures, where like numbers indicate like features.

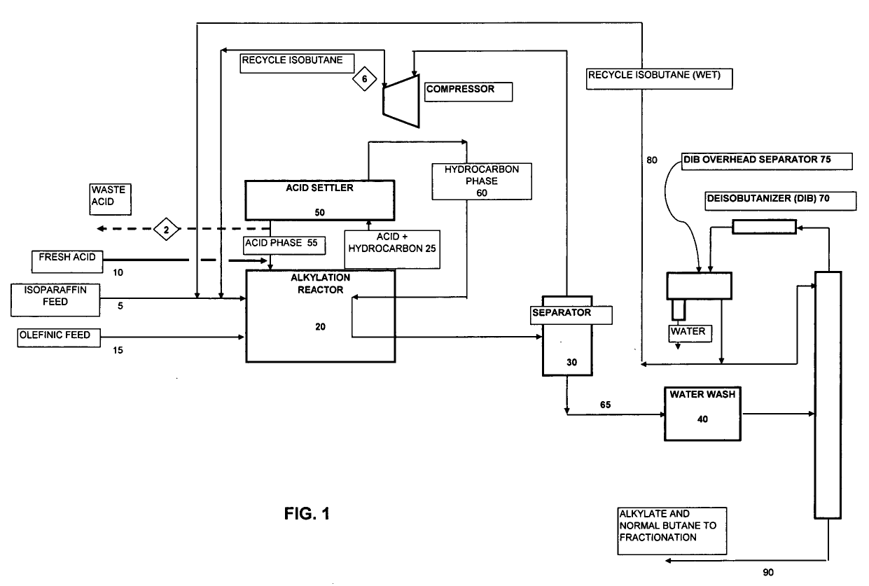

In the alkylation process shown generally in FIG. 1, an isoparaffin feed (5) and olefinic feed (15) are passed into an alkylation reactor (20) where they are contacted with an acid stream. The products of the reaction (25) are passed into an acid settler (50) where the hydrocarbon phase (60) is separated and the acid phase (55) is returned to the alkylation reactor. The hydrocarbon phase passes to a separator (30), the liquid phase (65) from which is washed (40) to remove trace acid and ester from the hydrocarbon. The washed hydrocarbon phase passes through a deisobutanizer column (DIB) (70) to separate it into an overhead product comprising primarily isobutane (80) and a bottoms stream containing normal butane and the alkylate product (90). The hydrocarbon feed to the DIB is saturated with water, which normally distills overhead. The isobutane recycle (80) is therefore typically saturated with water and may also contain undissolved water entrained from the deisobutanizer overhead separator (75). The overhead product of the distillation (80) is recycled to the alkylation reactor (20) to maintain a suitably high ratio of isobutane to olefin in the reactor (20). Recycle isobutane (6) from the compressor is passed back into the isoparaffin feed (5) using suitable means, as known in the art. Waste acid is withdrawn via stream 2.

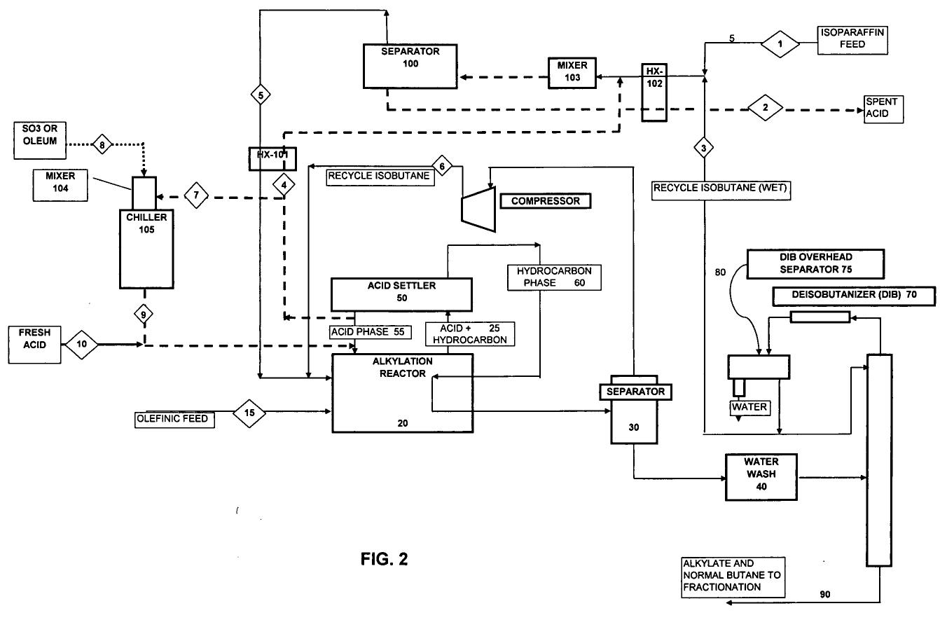

In the first method of the present invention (shown in FIG. 2) the acid withdrawn from the alkylation reactor (spent acid) (stream 4) is used to remove water from the isobutane recycle (stream 3) or any other hydrocarbon stream not containing olefin entering the reaction section, such as a stream of isobutane from an outside source (stream 1), before the hydrocarbon stream contacts the acid catalyst in the reactor system. Preferably, before it contacts the spent acid, the hydrocarbon stream will be dewatered by means of a coalescer/settler or other suitable means known to those of ordinary skill in the art (not shown). The withdrawn acid is mixed with the feed stream by means of a static mixer or other conventional mixing device (103) known in the art and then the hydrocarbon and acid are separated by settling in a separator (100). The acid is highly hydroscopic and can generally reduce the water in the hydrocarbon phase to below 50 ppmw in a simple mixer/settler contact. The spent acid (stream 2), containing the water removed from the feed streams, is removed from the alkylation unit for regeneration or other disposition and the dried hydrocarbon stream (stream 5) then passes to the alkylation reactor (20).

One example of the second method of the invention is also shown in FIG. 2. An SO₃-containing substance is added via stream 8 to a mixer 104 when it contacts acid 55 from settler 50 through stream 7. This produces fortified acid (stream 9). The temperature of the mixture is controlled to a desired temperature by chiller 105. Fresh acid 10 is added and the mixture is added to reactor 20. The proportion of SO₃, acid from Settler 50 and fresh acid containing less than 2%, and preferably 0.2 to 0.8% water is controlled so that the local concentration of water in the catalyst at any point is not less than 0.8%. Heat exchangers (HX) may be added at appropriate points in the process, as known in the art. Some non-limiting examples of heat exchangers are shown in the Figures. Other examples and placements of heat exchangers are known in the art.

The temperature of the fortified acid must be controlled below about 60° F. to minimize side reactions with the red oil present in the acid catalyst. These side reactions with red oil increase as the temperature rises, decreasing the effectiveness of the SO₃-containing substance in the catalyst water reduction and producing sulfonated hydrocarbons that are detrimental to the alkylation process. The reaction between SO₃ and water is highly exothermic. To keep the temperature below 60° F., either the temperature of the wet acid must be sufficiently below 60° F. so that the heat of reaction does not raise its temperature above 60° F., or the heat of reaction must be removed by heat exchange as the reaction occurs (as in chiller 105 in FIG. 2). Such heat removal may be carried out by conventional means known to those of ordinary skill in the art. An advantage of this process is that the heat of reaction between acid and free water is released and removed outside the alkylation reactor, eliminating the local temperature rise that would occur in the reactor as a consequence of that reaction taking place in the reactor.

The SO₃-containing substance used in the process of the invention may be one or more of oleum of various concentrations (such as 13%, 16%, 20%, 30% or 65%), or 100% liquid SO₃.

This method of removing water by reaction with SO₃ adds one degree of freedom in control of the two diluents of the acid system so that the red oil concentration can be controlled by the rate of acid withdrawal while the water concentration is controlled by reaction with SO₃.

Although Applicants do not wish to be bound by theory, an overall balance shows that at a constant rate of water carried by feed streams that enter the reactor and at constant rate of replacement of acid, the spent acid, after contact with the recycle isobutane and other wet hydrocarbon streams according to the present invention, will contain the same concentration of water as it would without the drying step of this invention, but according to this invention the acid acquires the water after the acid leaves the reactor and before the water enters it, so the concentration of water in the reactor is reduced by this invention. It is obvious to one skilled in the art that the reduction in water concentration in the catalyst at constant acid replacement rate can be traded for a reduction in acid replacement rate to achieve a new optimum economic balance between enhanced performance of the lower concentration of water in the acid catalyst and the cost of acid replacement. Among the benefits of operation of the process with a lower concentration of water in the catalyst are increased alkylate octane rating in the product and increased solubility of hydrocarbons such as isobutane in the acid catalyst, which reduces the rate of production of red oil. Another benefit of contacting the spent acid with isobutane is to complete reaction of sulfuric esters remaining in the acid phase. The esters are an intermediate in the alkylation reaction formed by reaction between sulfuric acid and olefins and then react further with isoparaffin to form alkylate and sulfuric acid. Trace quantities of ester in the acid phase remain unconverted in the alkylation reactor and the amount withdrawn in spent acid represents a loss of alkylate yield. Because the esters of C3 olefin are more stable than those of the C4 and C5 olefins, the optimum reactor temperature in a unit using C3 and heavier olefins as feed must be higher than the optimum temperature for C4 and or C5 olefin, in order to increase the rate of reaction or decomposition of the C3 ester to prevent the loss of ester to spent acid and to reduce the accumulation of ester in the acid phase. The higher temperature required for C3 alkylation adversely affects the alkylation of C4 and C5 olefin, reducing the alkylate octane number and increasing the rate of formation of red oil, forcing higher rate of acid replacement. The contacting of the spent acid with isobutane in the absence of olefin is known to effectively convert the ester to alkylate. Effecting that contact at a temperature higher than the alkylation reactor accelerates the reactions that convert the ester to alkylate. It follows that because the process of this invention affords a means of recovering the ester in the spent acid by completing its conversion of ester to alkylate, a reactor alkylating C3 olefin along with C4 or C4 and C5 olefins can be operated at a lower temperature than if the esters in spent acid were not recoverable, thereby improving alkylate yield and quality.

In a preferred embodiment, the isoparaffin feed is selected from C4 and C5 isoparaffins and the temperature and nature of mixing, including intensity, number of stages of contact, and whether cocurrent or countercurrent, of the drying contactor are optimized for conversion of esters in the acid phase to alkylate, which dissolves in the hydrocarbon phase and is carried therein to the alkylation reactor section. The temperature and nature of mixing are controlled by means known in the art.

Another advantage to a drying step is that the heat of reaction of the water with the acid is not released within the refrigerated reaction system, thereby reducing the refrigeration load on the process.

The use of non-contact heat exchange between isobutane or other hydrocarbon recycle and the spent acid controls the temperature of the hydrocarbon/acid drying contactor and reduces load on the reactor refrigeration system by utilizing the cold spent acid to cool the isobutane recycle or other hydrocarbon stream. As shown in FIG. 2, isobutane recycle may exchange heat with spent acid in a countercurrent flow upstream (HX-102) or downstream (HX-101), or both, of the spent acid/isoparaffin contactor (103) and separator (100). The extent of heat exchange in each heat exchanger is controlled by means known in the art (not shown).

In an alkylation unit fed with two or more separate streams having olefins of one carbon number each, the alkylate yield and quality are improved if the feed streams are directed to separate reaction zones such that each carbon number olefin reacts in the absence of other carbon number olefins and the reaction conditions, including temperature and the concentrations of red oil and water in the catalyst, can be optimized for each carbon number olefin. In such case, the fresh and fortified acid streams would be reintroduced to the reaction zones at points known to one of ordinary skill in the art, in order to adjust the composition of acid within each of those zones to optimize process performance. In such a process with multiple reactor/settler systems, the rates of withdrawal of acid to spending (4) and to fortification (7) and the rates of introduction of the fresh acid (10) and fortified acid (9) are preferably chosen to optimize the composition of the acid catalyst in each reaction zone. These chosen optimization rates are determinable by one of ordinary skill in the art without undue experimentation. The SO₃-containing substance may be mixed with each of two or more streams of acid circulated from different settlers to individual mixing devices and returned to different reactors, the rate of injection of SO₃ into each reactor system being selected to achieve the desired concentration of water in each reactor. The rate of withdrawal and replacement of acid at each reactor may also be controlled to optimize the concentration of red oil in that zone. The fresh acid, containing some free water and no red oil, and the fortified acid, containing a high concentration of red oil and minimal free water, may be directed independently to different points to provide the additional degrees of freedom in optimization of acid catalyst properties in the reaction system.

In one embodiment, the teachings of U.S. Pat. No. 3,803,262 are applied by adding to the system shown in FIG. 2 a contacting device wherein the olefinic feed stream 15 is contacted with at least a portion of the spent acid stream 4 in order to react the olefin with sulfuric acid to form dialkyl sulfates. Some of the dialkyl sulfates dissolve in the olefinic hydrocarbon phase and are carried to the alkylation reactor section. Other dialkyl sulfates formed in this olefin/acid contactor remain dissolved in the acid phase, which is then directed to the acid/isobutane contactor 103 provided according to the present invention to dry the isobutane. In this embodiment, the acid/isobutane contactor is designed to effect sufficient contact between the phases to effect the reaction of some of the alkyl sulfates to alkylate and sulfuric acid and extraction of unreacted dialkyl sulfates and some of the alkyl sulfates from the acid phase into the hydrocarbon phase that subsequently carries them to the reactor section. The alkyl and dialkyl sulfates carried by the olefin stream and the isobutane stream to the alkylation reactor further react with isoparaffin to form valuable alkylate product and sulfuric acid, effectively recovering pure sulfuric acid from the spent acid and thereby reducing the amount of fresh acid that must be discarded and replaced to control the concentration of red oil. The contact so described also effects the drying of the olefin and isobutane according to the teachings of the current invention.

The means of reducing dilution of the acid by water described herein may be applied to other systems, including for instance a mixture of nitric and sulfuric acids used for nitration of hydrocarbons, which reactions produce water as a byproduct, requiring replacement of the sulfuric acid to maintain adequate acid concentration. Other applications of the methods described herein will be obvious to one skilled in the art without undue experimentation using the description herein.

As known in the art, the concentrations of red oil and water of the sulfuric acid catalyst are optimally controlled at values dependent on the process in which sulfuric acid is used as a catalyst. The optimum water content of the sulfuric acid catalyst depends on many factors including the temperature of the process and the composition of the feedstock. Where the concentrations of red oil and water in the catalyst are both controlled by withdrawal and replacement of acid, the two variables cannot be controlled independently at their respective optimum values. The present invention allows independent control of the concentrations of both the water and the red oil in the sulfuric acid catalyst to their respective optimum values. The concentration of red oil is controlled by modulating the rate of withdrawal of sulfuric acid and the concentration of water is controlled by modulating the rate of addition of an SO₃-containing substance to the catalyst system by mixing with a stream of acid catalyst circulated to an external mixing device and back to the alkylation reactor.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

All patents and publications mentioned in the specification are indicative of the levels of skill of those skilled in the art to which the invention pertains. One skilled in the art would readily appreciate that the present invention is well adapted to carry out the objects and obtain the ends and advantages mentioned, as well as those inherent therein. The processes, methods and accessory methods described herein as presently representative of preferred embodiments are exemplary and are not intended as limitations on the scope of the invention. Changes therein and other uses will occur to those skilled in the art, which are encompassed within the spirit of the invention, are defined by the scope of the claims. Although the description herein contains many specificities, these should not be construed as limiting the scope of the invention, but as merely providing illustrations of some of the embodiments of the invention. Thus, additional embodiments are within the scope of the invention and within the following claims. For example, the water content of feed streams other than specifically described can be reduced by one of ordinary skill in the art using the methods described herein. All references cited herein are hereby incorporated by reference to the extent that there is no inconsistency with the disclosure of this specification. Some references provided herein are incorporated by reference herein to provide details concerning additional process steps and additional uses of the invention.

We claim:

1. A method for reducing the water content of a sulfuric acid catalyst in a process catalyzed by sulfuric acid, said process carried out in a reactor, said method comprising:
(a) continuously circulating a catalyst stream from the acid settler to a mixing area, forming a wet catalyst stream;
(b) continuously adding an SO₃-containing substance to the wet catalyst stream in the mixing area, forming a fortified catalyst stream, wherein the rate of circulation of the catalyst stream is selected to control the concentration of water in the fortified catalyst stream to at least about 0.8% by weight of the acid and the rate of addition of SO₃-containing substance is selected to control the concentration of water at one or more reaction zones to a selected value;
(c) maintaining the temperature of the catalyst stream during and after mixing below about 60° F.;
(d) returning the fortified catalyst stream to one or more zones of the reactor system.

2. The method of claim 1, wherein said selected value is between about 1.5 to about 4% by weight of acid.

3. The method of claim 1, wherein the rate of withdrawal of spent acid from the acid-catalyzed process and its replacement with fresh acid are modulated to control the concentration of red oil in the acid phase in the process.

4. The method of claim 1, wherein the SO₃-containing substance is oleum.

5. The method of claim 1 in which the process is an alkylation process wherein a hydrocarbon feed stream is contacted with an alkylating agent in the presence of a sulfuric acid catalyst under alkylation conditions in a reactor having one or more reaction zones, said reactor fluidly connected to an acid settler.

6. The method of claim 5, wherein the alkylating agent comprises one or more olefins.

7. The method of claim 5, wherein the alkyation process comprises two or more separate reactor systems with different hydrocarbon feed streams, each reactor having a separate acid settler, wherein catalyst streams are withdrawn from separate acid settlers, fortified separately, and returned to points in each reactor system to optimize the concentration of water in the separate alkylation reactors.

8. The method of claim 5, wherein the hydrocarbon stream comprises one or more paraffins.

9. A method for removing water from a liquid stream essentially immiscible and unreactive with sulfuric acid before it contacts the acid catalyst of a process catalyzed by sulfuric acid, said process carried out in a reactor, comprising:
(a) removing spent acid from the reactor;
(b) contacting the hydrocarbon stream with the spent acid in a drying contactor, forming a dried hydrocarbon stream and an acid phase;
(c) separating the dried hydrocarbon stream from the acid phase in a drying separator;
(d) sending the dried hydrocarbon stream to the reactor;
(e) removing the acid phase from the drying separator.

10. The method of claim 9, wherein the rate of withdrawal of spent acid from the acid-catalyzed process and its replacement with fresh acid are modulated to control the concentration of red oil in the acid phase in the process.

11. The method of claim 9 in which the process is an alkylation process wherein a hydrocarbon feed stream is contacted with an alkylating agent in the presence of a sulfuric acid catalyst under alkylation conditions in a reactor having one or more reaction zones, said reactor fluidly connected to an acid settler.

12. The method of claim 11, wherein the hydrocarbon stream comprises one or more paraffins and is essentially free of olefins.

13. The method of claim 11, wherein the hydrocarbon stream is a recycle isoparaffin stream.

14. A method for reducing the water content of a sulfuric acid catalyst in a sulfuric acid-catalyzed process fed by a liquid stream essentially immiscible and unreactive with sulfuric acid and carried out in a reactor, comprising:
(a) continuously circulating a catalyst stream from the acid settler to a mixing area, forming a wet catalyst stream;
(b) continuously adding an SO₃-containing substance to the wet catalyst stream in the mixing area, forming a fortified catalyst stream, wherein the rate of circulation of the catalyst stream is selected to control the concentration of water in the fortified catalyst stream to at least about 0.8% by weight of the acid and the rate of addition of SO₃-containing substance is selected to control the concentration of water at one or more reaction zones to a selected value;
(c) maintaining the temperature of the catalyst stream during and after mixing below about 60° F.;
(d) returning the fortified catalyst stream to one or more zones of the reactor system.
(e) removing spent acid from the reactor;
(f) contacting the liquid feed stream with the spent acid in a drying contactor, forming a dried liquid feed phase and an acid phase;
(g) separating the dried liquid feed stream from the acid phase in a drying separator;
(h) sending the dried liquid feed stream to the reactor;
(i) removing the acid phase from the drying separator.

15. The method of claim 14, wherein the SO₃-containing substance is oleum.

16. The method of claim 14, wherein the acid phase in the reactor after return of the fortified catalyst stream contains between 1.5 to 4% water by weight.

17. The method of claim 14, wherein the rate of withdrawal of spent acid from the acid-catalyzed process and its replacement with fresh acid are modulated to control the concentration of red oil in the acid phase in the process.

18. The method of claim 14 in which the process is an alkylation process wherein a hydrocarbon feed stream is contacted with an alkylating agent in the presence of a sulfuric acid catalyst under alkylation conditions in a reactor having one or more reaction zones, said reactor fluidly connected to an acid settler.

19. The method of claim 18, wherein the hydrocarbon stream comprises one or more paraffins essentially free of olefin.
